(19) United States
(12) Patent Application Publication
(10) Pub. No.: US 2019/0015660 A1
(43) Pub. Date: Jan. 17, 2019

(54) SYSTEMS AND METHODS FOR PLANNING AND PROGRAMMING ELECTRICAL STIMULATION

(71) Applicant: Boston Scientific Neuromodulation Corporation, Valencia, CA (US)

(72) Inventors: Tianhe Zhang, Studio City, CA (US); Michael A. Moffitt, Saugus, CA (US)

(21) Appl. No.: 16/031,478

(22) Filed: Jul. 10, 2018

Related U.S. Application Data
(60) Provisional application No. 62/532,860, filed on Jul. 14, 2017.

(51) Int. Cl. A61N 1/05; G06T 7/11; A61N 1/372; G06T 7/30; G06K 9/00; A61B 5/00

(52) U.S. Cl. CPC A61N 1/0529; G06T 7/11; A61B 5/7475; G06T 7/30; G06K 9/00362; A61N 1/37241

(57) ABSTRACT

A system for planning implantation of an electrical stimulation lead of an electrical stimulation system and programming the electrical stimulation system after implantation includes a display; and a processor that executes instructions to perform actions, including: receiving an anatomical target or etiology for stimulation; receiving at least one pre-implantation image; after implantation of the electrical stimulation lead, receiving an estimate of the implantation site; presenting a display of a model of the electrical stimulation lead and anatomical features based on the estimate of the implantation site; and providing an initial set of stimulation parameters for programming the electrical stimulation system based on the estimate of the implantation site.

### CROSS-REFERENCE TO RELATED APPLICATIONS

This application claims the benefit under 35 U.S.C. § 119(e) of U.S. Provisional Patent Application Ser. No. 62/532,860, filed Jul. 14, 2017, which is incorporated herein by reference.

### FIELD

The present invention is directed to the area of implantable electrical stimulation systems and methods of making, using, and programming the systems. The present invention is also directed to interfaces for diagnosing, planning, or programming electrical stimulation systems.

### BACKGROUND

Implantable electrical stimulation systems have proven therapeutic in a variety of diseases and disorders. For example, stimulation of the brain, such as deep brain stimulation, can be used to treat a variety of diseases or disorders and spinal cord stimulation systems have been used as a therapeutic modality for the treatment of chronic pain syndromes. Peripheral nerve stimulation has been used to treat incontinence, as well as a number of other applications under investigation. Functional electrical stimulation systems have been applied to restore some functionality to paralyzed extremities in spinal cord injury patients.

Stimulators have been developed to provide therapy for a variety of treatments. A stimulator can include a control module (with a pulse generator), one or more leads, and an array of stimulator electrodes on each lead. The stimulator electrodes are in contact with or near the brain, nerves, or other tissue to be stimulated. The pulse generator in the control module generates electrical pulses that are delivered by the electrodes to body tissue.

### BRIEF SUMMARY

One embodiment is a system for planning implantation of an electrical stimulation lead of an electrical stimulation system and programming the electrical stimulation system after implantation. The system includes a display; and a processor that executes instructions to perform actions, including: receiving an anatomical target for stimulation; receiving at least one pre-implantation image including the anatomical target; receiving or determining a target implantation site based on the anatomical target; providing an indication of the target implantation site on the display to aid implantation of the electrical stimulation lead; after implantation of the electrical stimulation lead, receiving or determining an estimate of the actual implantation site; and providing an initial set of stimulation parameters for programming the electrical stimulation system based on the estimate of the actual implantation site and the anatomical target.

In at least some embodiments, the instructions further include receiving results of trial stimulation conducted following the implantation, wherein providing an initial set of stimulation parameters includes providing the initial set of stimulation parameters for programming the electrical stimulation site based on the estimate of the actual implantation site, the anatomical target, and the results of the trial stimulation. In at least some embodiments, the results of the trial stimulation include at least one set of trial stimulation parameters and, for at least one of the at least one set of trial stimulation parameters, an indication of a clinical result obtained when using that set of trial stimulation parameters.

In at least some embodiments, the instructions further including receiving or determining a registration of the at least one pre-implantation image to an anatomical atlas. In at least some embodiments, receiving an anatomical target for stimulation includes receiving an etiology and determining the anatomical target for stimulation based on the etiology. In at least some embodiments, receiving an anatomical target for stimulation includes receiving a target stimulation effect and determining the anatomical target for stimulation based on the target stimulation effect. In at least some embodiments, the instructions further including receiving at least one post-implantation image of the anatomical site and the implanted electrical stimulation lead.

Another embodiment is a system for planning implantation of an electrical stimulation lead of an electrical stimulation system and programming the electrical stimulation system after implantation. The system includes a display; and a processor that executes instructions to generate a user interface, the user interface including: one or more input controls for inputting patient information; a target region for inputting or selecting an anatomical target or etiology for stimulation; an image region for displaying at least one pre-implantation image including the anatomical target or anatomical region associated with the etiology; an implantation site region for inputting or selecting an actual, estimated, or planned implantation site for a stimulation lead; an estimate region for displaying an estimate of a position of the stimulation lead at the actual, estimated or planned implantation site relative to one or more anatomical features; and a programming region for inputting or modifying stimulation parameters for programming the electrical stimulation system for delivery of electrical stimulation using the electrical stimulation lead.

In at least some embodiments, the user interface further includes a note region for inputting notes by the user regarding implantation or programming. In at least some embodiments, the estimate region further includes displaying, relative to the stimulation lead, an estimated region of stimulation based on a set of stimulation parameters. In at least some embodiments, the processor is configured and arranged to alter the estimated region of stimulation when the stimulation parameters are modified.

In at least some embodiments, the user interface further includes an atlas registration region for accessing an anatomical atlas. In at least some embodiments, the processor is configured to register the anatomical atlas to the at least one pre-implantation image. In at least some embodiments, the image region is further configured for displaying at least one post-implantation image.

Yet another embodiment is a system for planning implantation of an electrical stimulation lead of an electrical stimulation system and programming the electrical stimulation system after implantation. The system includes a display; and a processor that executes instructions to perform actions, including: receiving an anatomical target or etiology for stimulation; receiving at least one pre-implantation image; after implantation of the electrical stimulation lead, receiving an estimate of the implantation site; presenting a display of a model of the electrical stimulation lead and anatomical features based on the estimate of the implantation site; and providing an initial set of stimulation parameters for programming the electrical stimulation system based on the estimate of the implantation site.

In at least some embodiments, the instructions further include receiving results of trial stimulation conducted following the implantation, wherein providing an initial set of stimulation parameters includes providing the initial set of stimulation parameters for programming the electrical stimulation site based on the estimate of the actual implantation site, the anatomical target, and the results of the trial stimulation. In at least some embodiments, the results of the trial stimulation include at least one set of trial stimulation parameters and, for at least one of the at least one set of trial stimulation parameters, an indication of a clinical result obtained when using that set of trial stimulation parameters.

In at least some embodiments, the instructions further including receiving or determining a registration of the at least one pre-implantation image to an anatomical atlas. In at least some embodiments, receiving an anatomical target or etiology for stimulation includes receiving the etiology and determining the anatomical target for stimulation based on the etiology. In at least some embodiments, the instructions further including receiving at least one post-implantation image of the anatomical site and the implanted electrical stimulation lead.

Yet other embodiments include a non-transitory computer-readable medium having computer executable instructions stored thereon that, when executed by at least one processor, cause the at least one processor to perform the actions described above for any one of the embodiments. Further embodiments include a non-transitory computer-readable medium having computer executable instructions stored thereon that, when executed by at least one processor, cause the at least one processor to generate the user interface described above for any one of the embodiments.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic view of one embodiment of an electrical stimulation system, according to the invention;

FIG. 2A is a schematic side view of some embodiments of a distal portion of an electrical stimulation lead, according to the invention;

FIG. 2B is a schematic side view of a second embodiment of a distal portion of an electrical stimulation lead, according to the invention;

FIG. 2C is a schematic side view of a third embodiment of a distal portion of an electrical stimulation lead, according to the invention;

FIG. 2D is a schematic side view of a fourth embodiment of a distal portion of an electrical stimulation lead, according to the invention;

FIG. 2E is a schematic side view of a fifth embodiment of a distal portion of an electrical stimulation lead, according to the invention;

FIG. 2F is a schematic side view of a sixth embodiment of a distal portion of an electrical stimulation lead, according to the invention;

FIG. 2G is a schematic side view of a seventh embodiment of a distal portion of an electrical stimulation lead, according to the invention;

FIG. 2H is a schematic side view of an eighth embodiment of a distal portion of an electrical stimulation lead, according to the invention;

FIG. 2I is a schematic side view of a ninth embodiment of a distal portion of an electrical stimulation lead, according to the invention;

FIG. 3 is a schematic view of a workflow for planning, implanting, and programming an electrical stimulation system;

FIG. 4 is a schematic block diagram of one embodiment of a system for planning, implanting, and programming an electrical stimulation system, according to the invention;

FIG. 5 is one embodiment of a planning portion of a user interface, according to the invention;

FIG. 6 is one embodiment of an estimate portion of a user interface, according to the invention;

FIG. 7 is one embodiment of a programming portion of a user interface, according to the invention;

FIG. 8 is another embodiment of a programming portion of a user interface, according to the invention; and

FIG. 9 is a schematic block diagram of a work flow and components for planning, implanting, and programming an electrical stimulation system, according to the invention.

### DETAILED DESCRIPTION

The present invention is directed to the area of implantable electrical stimulation systems and methods of making, using, and programming the systems. The present invention is also directed to interfaces for diagnosing, planning, or programming electrical stimulation systems.

Suitable implantable electrical stimulation systems include, but are not limited to, an electrode lead (“lead”) with one or more electrodes disposed on a distal end portion of the lead and one or more terminals disposed on one or more proximal end portions of the lead. Leads include, for example, deep brain stimulation leads, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734;7,761,165; 7,783,359; 7,792,590; 7,809,446; 7,949,395; 7,974,706; 8,175,710; 8,224,450; 8,271,094; 8,295,944; 8,364,278; 8,391,985; and 8,688,235; and U.S. Patent Applications Publication Nos. 2007/0150036; 2009/0187222; 2009/0276021; 2010/0076535; 2010/0268298; 2011/0005069; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; 2013/0105071; and 2013/0197602, all of which are incorporated by reference.

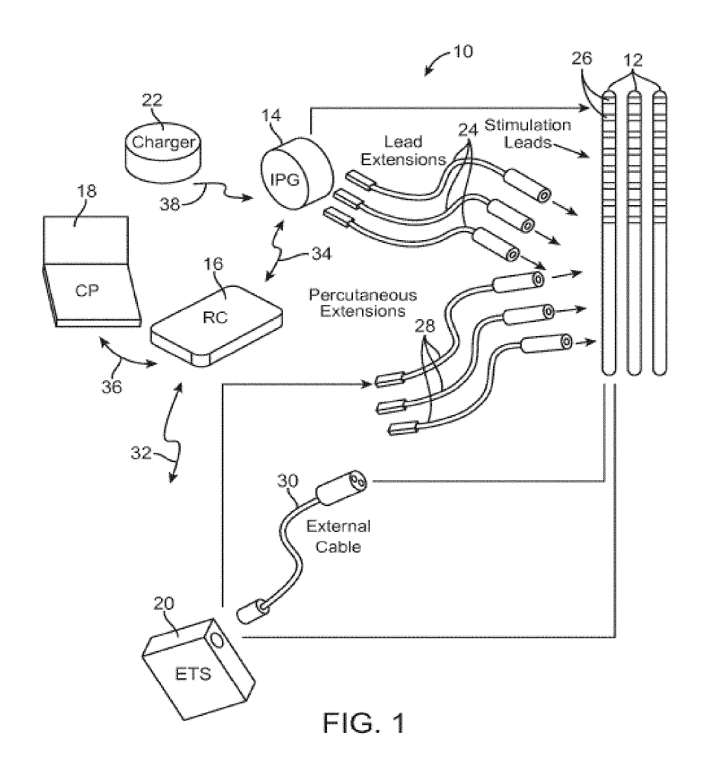

Turning to FIG. 1, one embodiment of an electrical stimulation system 10 includes at least one stimulation lead 12 and an implantable pulse generator (IPG) 14. The system 10 can also include at least one of an external remote control (RC) 16, a clinician's programmer (CP) 18, an external trial stimulator (ETS) 20, or an external charger 22. The IPG 14 and ETS 20 can be considered control modules.

The IPG 14 is physically connected, optionally via at least one lead extension 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The IPG 14 can have eight or more stimulation channels which may be independently programmable to control the magnitude of the current stimulus from each channel. In at least some embodiments, the IPG 14 can have more or fewer than eight stimulation channels (for example, 4, 6, 16, 32, or more stimulation channels). The IPG 14 can have one, two, three, four, or more connector ports, for receiving the terminals of the leads.

The ETS 20 may also be physically connected, optionally via the percutaneous lead extensions 28 and external cable 30, to the stimulation leads 12. The ETS 20, which may have similar pulse generation circuitry as the IPG 14, also delivers electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test functioning of the system or the responsiveness of the stimulation that is to be provided. Any functions described herein with respect to the IPG 14 can likewise be performed with respect to the ETS 20.

The RC 16 may be used to telemetrically communicate with or control the IPG 14 or ETS 20 via a uni- or bi-directional wireless communications link 32. Once the IPG 14 and neurostimulation leads 12 are implanted, the RC 16 may be used to telemetrically communicate with or control the IPG 14 via a uni- or bi-directional communications link 34. Such communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions.

The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is expressly incorporated herein by reference. Other examples of electrical stimulation systems can be found at U.S. Pat. Nos. 6,181,969; 6,516,227; 6,609,029; 6,609,032; 6,741,892; 7,949,395; 7,244,150; 7,672,734; and 7,761,165; 7,974,706; 8,175,710; 8,224,450; and 8,364,278; and U.S. Patent Application Publication No. 2007/0150036, as well as the other references cited above, all of which are incorporated by reference.

The electrodes 26 of the lead 12 can be formed using any conductive, biocompatible material. Examples of suitable materials include metals, alloys, conductive polymers, conductive carbon, and the like, as well as combinations thereof. The number of electrodes 26 on the lead 12 may vary. For example, there can be two, three, four, five, six, seven, eight, nine, ten, eleven, twelve, thirteen, fourteen, fifteen, sixteen, or more electrodes. In the illustrated leads 12 of FIG. 1, the electrodes 26 are ring electrodes. Any number of ring electrodes can be disposed along the length of the lead body including, for example, one, two three, four, five, six, seven, eight, nine, ten, eleven, twelve, thirteen, fourteen, fifteen, sixteen or more ring electrodes. It will be understood that any number of ring electrodes can be disposed along the length of the lead body 111.

Terminals are typically disposed at the proximal end portion of the lead body 111 for connection to corresponding conductive contacts in one or more connector assemblies disposed on, for example, the IPG 14 or lead extension 28 or external cable 30 (or to other devices, such as conductive contacts on a splitter, an adaptor, or the like).

Conductive wires extend from the plurality of terminals to the array of electrodes 26. Typically, each of the plurality of terminals is electrically coupled to at least one electrode 26 of the array of electrodes. In at least some embodiments, each of the plurality of terminals is coupled to a single electrode 26 of the array of electrodes.

The lead body 111 forms a jacket around portions of the lead between the electrodes and terminals. The lead body 111 is typically a non-conductive, biocompatible material including, for example, silicone, polyurethane, polyetheretherketone (“PEEK”), epoxy, perfluoroalkoxy alkane (PFA), and the like or combinations thereof. The lead body 111 may be formed in the desired shape by any process including, for example, extruding, molding (including injection molding), casting, and the like. Electrodes 26 and conductor wires can be disposed onto or within the lead body 111 either prior to or subsequent to a molding or casting process. The non-conductive material typically extends from the distal end portion of the lead body 111 to the proximal end portion of the lead body 111.

The conductive wires may be embedded in the non-conductive material of the lead or can be disposed in one or more lumens (not shown) extending along the lead. In at least some embodiments, there is an individual lumen for each conductive wire. In other embodiments, two or more conductive wires may extend through a lumen. There may also be one or more lumens (not shown) that open at, or near, the proximal end portion of the lead 12, for example, for inserting a stylet rod to facilitate placement of the lead 12 within a body of a patient. Additionally, there may also be one or more lumens (not shown) that open at, or near, the distal end portion of the lead 12, for example, for infusion of drugs or medication into the site of implantation of the lead 12. The one or more lumens may, optionally, be flushed continually, or on a regular basis, with saline or the like. The one or more lumens can be permanently or removably sealable at the distal end portion.

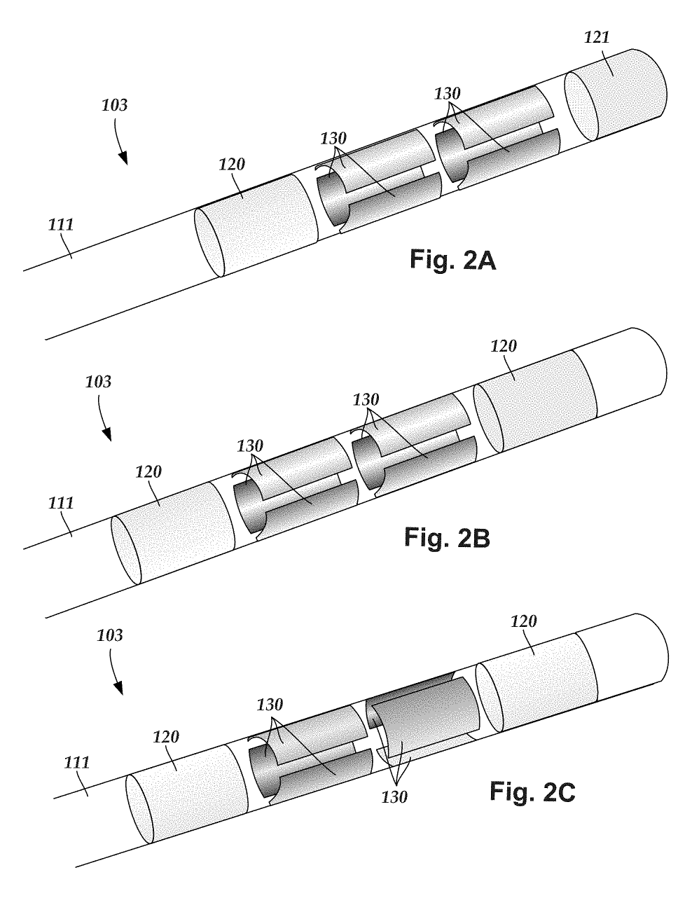

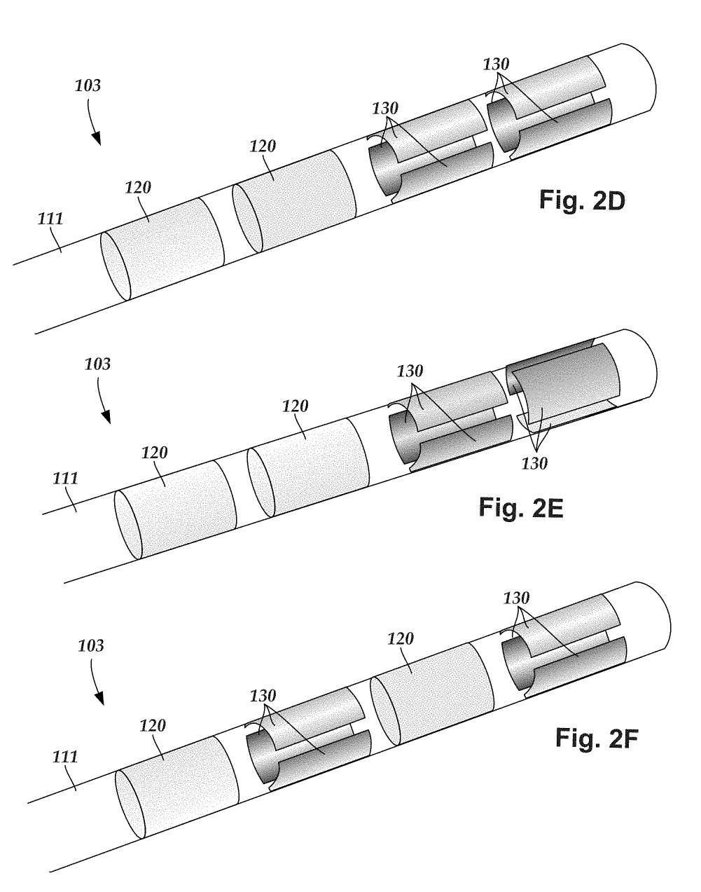

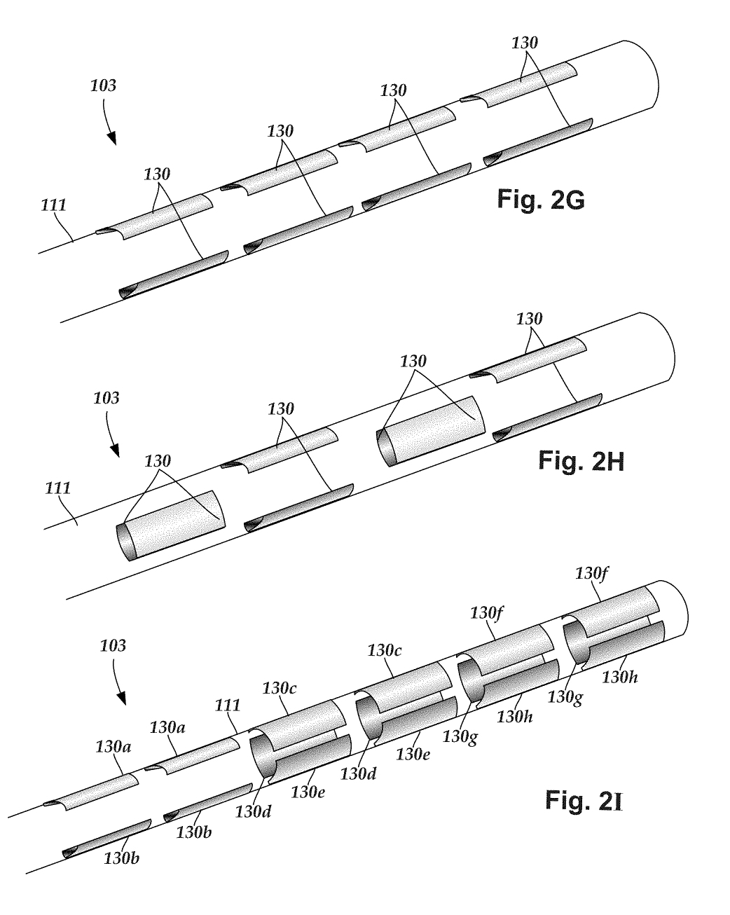

A lead can include ring electrodes, segmented electrodes, tip electrodes, or any other suitable electrode or any combination thereof. A lead containing ring electrodes and segmented electrodes may be arranged in any suitable configuration. FIGS. 2A-2I illustrate a variety of different arrangements as non-limiting examples. The arrangements can include ring electrodes 120; segmented electrodes 130, 130a-130h; or tip electrodes 121 disposed along a lead body 111 of a lead 103. FIG. 2A schematically illustrates a distal end portion of a lead 103 with a ring electrode 120, a tip electrode 121, and six segmented electrodes 130 in the distal electrode array 133. Segmented electrodes 130 may provide for superior current steering than ring electrodes 120 because target structures may not be disposed symmetrically about a longitudinal axis of the distal electrode array 133. Instead, a target may be located on one side of a plane running through the axis of the lead 103.

Through the use of a radially segmented electrode array (“RSEA”), current steering can be performed not only along a length of the lead 103 but also around a circumference of the lead 103. This provides precise three-dimensional targeting and delivery of the current stimulus to target tissue, while potentially avoiding stimulation of other tissue. Examples of leads with segmented electrodes include U.S. Patent Applications Publication Nos. 2010/0268298; 2011/0005069; 2011/0078900; 2011/0130803; 2011/0130816; 2011/0130817; 2011/0130818; 2011/0078900; 2011/0238129; 2011/0313500; 2012/0016378; 2012/0046710; 2012/0071949; 2012/0165911; 2012/197375; 2012/0203316; 2012/0203320; 2012/0203321; 2013/0197602; 2013/0261684; 2013/0325091; 2013/0317587; 2014/0039587; 2014/0353001; 2014/0358209; 2014/0358210; 2015/0018915; 2015/0021817; 2015/0045864; 2015/0021817; 2015/0066120; 2013/0197424; 2015/0151113; 2014/0358207; and U.S. Pat. No. 8,483,237, all of which are incorporated herein by reference in their entireties. Examples of leads with tip electrodes include at least some of the previously cited references, as well as U.S. Patent Applications Publication Nos. 2014/0296953 and 2014/0343647, all of which are incorporated herein by reference in their entireties. A lead with segmented electrodes may be a directional lead that can provide stimulation in a particular direction using the segmented electrodes.

Any number of segmented electrodes 130 may be disposed on the lead body 111 including, for example, one, two three, four, five, six, seven, eight, nine, ten, eleven, twelve, thirteen, fourteen, fifteen, sixteen or more segmented electrodes 130. It will be understood that any number of segmented electrodes 130 may be disposed along the length of the lead body 111. A segmented electrode 130 typically extends only 75%, 67%, 60%, 50%, 40%, 33%, 25%, 20%, 17%, 15%, or less around the circumference of the lead.

The segmented electrodes 130 may be grouped into sets of segmented electrodes 130, where each set is disposed around a circumference of the lead 103 at a particular longitudinal portion of the lead 103. The lead 103 may have any number segmented electrodes 130 in a given set of segmented electrodes 130. The lead 103 may have one, two, three, four, five, six, seven, eight, or more segmented electrodes 130 in a given set. The segmented electrodes 130 may vary in size and shape. In at least some embodiments, the segmented electrodes 130 are all of the same size, shape, diameter, width or area or any combination thereof. In at least some embodiments, the segmented electrodes 130 of each circumferential set (or even all segmented electrodes disposed on the lead 103) may be identical in size and shape.

Each set of segmented electrodes 130 may be disposed around the circumference of the lead body 111 to form a substantially cylindrical shape around the lead body 111. The spacing between individual segmented electrodes 130 of a given set of the segmented electrodes 130 may be the same, or different from, the spacing between individual electrodes of another set of segmented electrodes 130 on the lead 103. In at least some embodiments, equal spaces, gaps or cutouts are disposed between each segmented electrode 130 around the circumference of the lead body 111. In other embodiments, the spaces, gaps or cutouts between the segmented electrodes 130 may differ in size or shape. In other embodiments, the spaces, gaps, or cutouts between segmented electrodes 130 may be uniform for a particular set of the segmented electrodes 130, or for all sets of the segmented electrodes 130. The sets of segmented electrodes 130 may be positioned in irregular or regular intervals along a length the lead body 111.

In at least some instances, arrangements of electrodes can be written in a shorthand, starting from the distal end portion, with each number indicating the number of electrodes at a particular longitudinal position. For example, the arrangement 1-3-3-1, illustrated in FIG. 2B, indicates a ring electrode 120 at the distal-most position, three segmented electrodes 130 at the next position, another three segmented electrodes 130 at the third position, and a ring electrode 120 at the proximal-most position. In addition, if there are multiple, sequential arrangements of the same type “×” can be used. As an example, the arrangement 3×5−1 (or 3×5+1) indicates five sets of three electrodes spaced apart longitudinally starting from the distal end portion with a single ring electrode 120 at the proximal-most position. Using this notation, the arrangements of the FIGS. 2A-2I can be written as follows: FIG. 2A: 1-3-3-1; FIG. 2B: 1-3-3-1; FIG. 2C: 1-3-3-1; FIG. 2D: 3-3-1-1; FIG. 2E: 3-3-1-1; FIG. 2F: 3-1-3-1; FIG. 2G: 2×4 (or 2-2-2-2); FIG. 2H: 2×4 (or 2-2-2-2); and FIG. 2I: 3×4-2×2 (or 3-3-3-3-2-2).

As non-limiting illustrations of electrode arrangements, when the lead 103 includes two ring electrodes 120 and two sets of segmented electrodes 130, the ring electrodes 120 can flank the two sets of segmented electrodes 130 (see, for example, FIGS. 2B and 2C). Alternately, the two ring electrodes 120 can be disposed proximal to the two sets of segmented electrodes 130 (see, for example, FIG. 2D and 2E), or the two ring electrodes 120 can be disposed distal to the two sets of segmented electrodes 130 (not shown) or the two ring electrodes 120 and two sets of segmented electrodes 130 can alternate (see, for example, FIG. 2F). An arrangement may also include a tip electrode 121 (see, for example, FIG. 2A) or a single ring electrode 120 either proximal to, distal to, or between the segmented electrodes (not shown). In arrangements with more than two sets of segmented electrodes 130, the segmented electrodes 130 of the sets may be aligned (see, for example, FIGS. 2A, 2B, 2D, 2F, 2G, and 21) or staggered (see, for example, FIGS. 2C, 2E, and 2H) relative to each other or in any other suitable relative arrangement. By varying the location of the segmented electrodes 130, different coverage of the target neurons may be selected. For example, the electrode arrangements of FIG. 2D or 2E may be useful if the physician anticipates that the neural target will be closer to a distal tip of the lead body 111. Any combination of ring electrodes 120, tip electrode 121, and segmented electrodes 130 may be disposed on the lead 103.

In at least some embodiments, the lead 103 may only include segmented electrodes 130. For example, FIGS. 2G and 2H illustrate the lead 103 with four pairs of segmented electrodes 130 (for example, a 2×4 arrangement) in aligned (FIG. 2G) or staggered configurations (FIG. 2H). Another arrangement has eight pairs of segmented electrodes 130 (for example, a 2×8 arrangement—not shown) in aligned or staggered configuration. FIG. 2I illustrates an arrangement in which different types of sets of segmented electrodes 130a-130h includes—in this case—four sets of three segmented electrodes 130c-130h and two pairs of segmented electrodes 130a, 130b (a 3×4-2×2 arrangement). Another example of a lead with segmented electrodes has the arrangement 3-3-2-3-2-3.

One variation of the arrangement of the lead 103 of FIG. 2I is to electrically gang (i.e., electrically short) segmented electrodes 130a-130h having the same reference numbers (for example, electrically gang the two segmented electrodes labeled 130a, etc.) Such electrical ganging can be accomplished in any suitable manner including by a conductor attached to two electrodes within the lead 100 or be electrically coupling the two electrodes to the same channel in the control module. Two, three, or more electrodes can be ganged together. The ganged electrodes provide longer virtual electrodes. In at least some embodiments, the ganged electrodes have an advantage, over very long individual contacts, of maintaining array flexibility while creating a longer virtual electrode. The ganged configuration maintains directionality and array span. Any other arrangement, including any of the arrangements illustrated in FIGS. 2A-21, can include two or more sets of electrically ganged electrodes. In at least some embodiments, a lead can include electrodes that are electrically ganged and other electrodes that are not ganged together.

In at least some embodiments, one or more electrodes include surface features to increase surface area of the electrodes. Examples of such surface features include dimples, scores, cuts, trenches, grooves, channels, knurls, or other depressions or roughening of the surface.

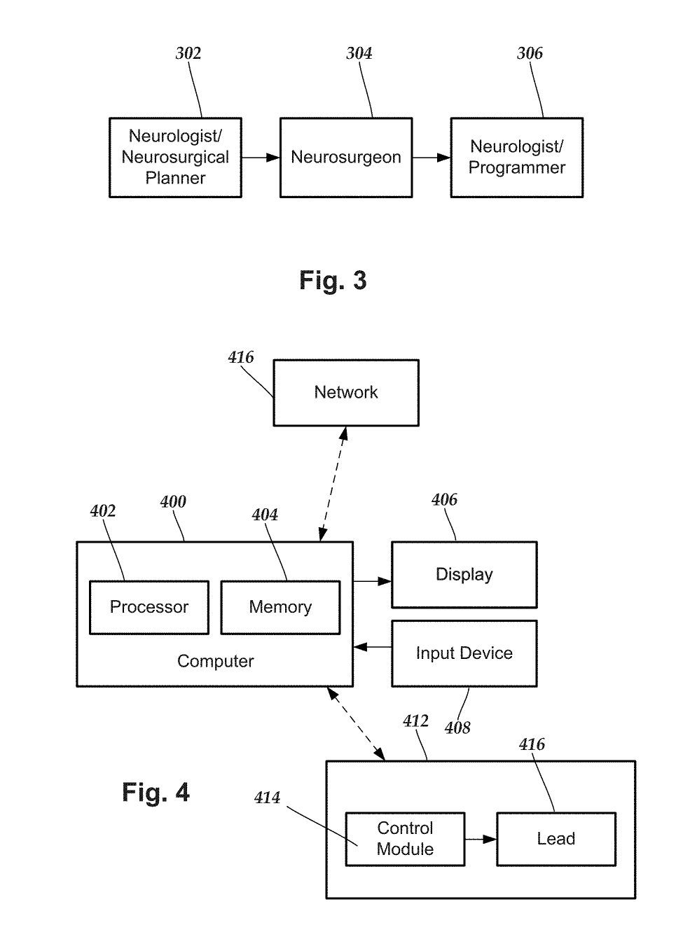

The process of implanting and programming an implantable electrical stimulation lead, such as a deep brain stimulation lead, includes multiple steps that may be performed by different individuals. FIG. 3 illustrates one example of a workflow. In 302, a neurologist or neurosurgical planner diagnoses the condition or disorder that is to be treated, develops a treatment plan, and refers the patient to a neurosurgeon. In some embodiments, the neurologist or neurosurgical planner selects a target or site for stimulation based on the diagnosis. In other embodiments, the neurologist or neurosurgical planner may utilize a system or software that proposes a target or site for stimulation which may be accepted, modified, or rejected by the neurologist or neurosurgical planner.

In 304, the neurosurgeon plans the surgery, implants the lead, and may perform an initial trial stimulation. In some embodiments, the neurosurgeon selects the stimulation site or implantation site based on the earlier diagnosis. In other embodiments, the stimulation site or implantation site may be suggested to the neurosurgeon and may be accepted, rejected, or modified by the neurosurgeon. In at least some embodiments, the neurosurgeon (or neurologist or neurosurgical planner) obtains pre-procedure imaging of the implantation or stimulation site. In some embodiments, the neurosurgeon (or neurologist or neurosurgical planner) may register the image(s) obtained from pre-procedure imaging to an anatomical atlas. Examples of atlas registration can be found at, for example, U.S. Pat. No. 8,675,945; and U.S. Patent Application Publications Nos. 2009/0118635; 2012/0314919; 2012/0314924; 2012/0330374; 2013/0039550; and 2015/0066111, all of which are incorporated herein by reference. In some embodiments, registration may also be based on trial stimulations.

In some embodiments, the system may suggest an implantation site. For example, the system (or other database or system) may utilize machine learning or other algorithms with data from previous patients or data from trial stimulations to select an implantation site based on the diagnosis, etiology, or the like. In some embodiments, the data used for machine learning or other algorithms may be selected from information gathered by the local surgical institution or, alternatively, may be obtained from multiple institutions using, for example, an accessible database where such information is stored. Advantages of limiting the information to that from a local surgical institution is that the available systems and technology and workflow procedures are likely to be relatively uniform. On the other hand, information from multiple institutions can be beneficial due to its quantity and the breadth of experience and different workflows, methodologies, and the like.

The neurosurgeon performs the implantation and may obtain post-procedure image(s) and may conduct a trial stimulation using the implanted lead. Once the stimulation lead (or a trial stimulation lead) is implanted, the neurosurgeon can provide notes or other indications (such as images or the like) regarding the site the implantation and, optionally, the results of a trial stimulation.

In 306, the same or different neurologist or a programmer then conducts one or more programming sessions to identify one or more sets of stimulation parameters or stimulation programs for therapeutic treatment using the implanted stimulation lead. Typically, the programming is performed manually by the neurologist or programmer and includes testing different sets of stimulation parameters or stimulation programs and identifying the results of the stimulation. In some instances, the neurologist or programmer may utilize systems or software that provide programming suggestions (for example, one or more suggested parameter sets) or automate (or partially automate) the programming of the lead by investigating different parameter sets.

It will be recognized that these activities may be distributed differently than described in this example or certain activities may be shared between the neurologist, neurosurgeon, and programmer. In addition, the selection of the site of implantation or the site of stimulation (or both) may be made by the neurologist, neurosurgical planner, neurosurgeon, or any combination thereof. As one example, the neurologist or neurosurgical planner may select or suggest a site of stimulation and the neurosurgeon may then select the site of implantation and develop a surgical plan for implanting the neurostimulation lead. As another example, the neurologist or neurosurgical planner may select or suggest a particular treatment result and the neurosurgeon may then select the site of stimulation and implantation and develop a surgical plan for implanting the neurostimulation lead.

A challenge in this process is the conveyance of information between the different individuals (e.g., neurologist and neurosurgeon) involved in the procedure. Conventionally, notes, verbal or written communication, and the like are used to convey information between the individuals. This information, however, may be incomplete and may not be provided in a systematic or consistent manner.

To better facilitate the communication of information between the different individuals involved in the planning, implantation, and programming of an electrical stimulation system, a user interface and accompanying software/hardware can be used by all of the individuals to provide comprehensive and uniform information for the procedure. The user interface allows the neurologist or neurosurgical planner to indicate the diagnosis and treatment plan; the neurosurgeon (or other person) to plan the surgical implantation and input information after surgery indicating the final placement of the electrical stimulation lead, as well as any initial stimulation testing; and the neurologist or programmer to conduct the programming session(s).

In some embodiments, the user interface and software are provided on several different devices which then communicate information to each other (or store information for later retrieval by the same or different device) through a wired or wireless network including, but not limited to, a local area network (LAN), a wide area network (WAN), a cloud network, the Internet, or any other suitable type of network. The information may be stored on one of the devices, on a storage medium (for example, a USB drive. DVD, or hard drive), on a separate server or storage device, in a cloud storage arrangement, or any other suitable, accessible storage device or arrangement, or any combination thereof. In other embodiments, the user interface and software may be provided on a single device (for example, a clinician programmer or a patient programmer) that is shared by the individuals.

FIG. 4 illustrates one embodiment of a system for practicing the invention. The system can include a computer 400 or any other similar device that includes a processor 402, a memory 404, a display 406, an input device 408, and, optionally, the electrical stimulation system 412 and a network 416. The network 416 can be a wired or wireless network such as, but not limited to, a local area network (LAN), a wide area network (WAN) a cloud network, the Internet, or any other suitable type of network. As described in the preceding paragraph, there may be multiple computers (of which computer 400 is an example) that are networked together or otherwise sharing information (for example, through the cloud, Internet, or a storage medium).

The computer 400 can be a laptop computer, desktop computer, tablet, mobile device, smartphone or other devices that can run applications or programs, or any other suitable device for processing information and for presenting a user interface. The computer can be, for example, a clinician programmer, patient programmer, or remote control for the electrical stimulation system 412. The computer 400 can be local to the user or can include components that are non-local to the user including one or both of the processor 402 or memory 404 (or portions thereof). For example, in some embodiments, the user may operate a terminal that is connected to a non-local computer. In other embodiments, the memory can be non-local to the user.

The computer 400 can utilize any suitable processor 402 including one or more hardware processors that may be local to the user or non-local to the user or other components of the computer. The processor 402 is configured to execute instructions provided to the processor, as described below.

Any suitable memory 404 can be used for the computer 402. The memory 404 illustrates a type of computer-readable media, namely computer-readable storage media. Computer-readable storage media may include, but is not limited to, nonvolatile, non-transitory, removable, and non-removable media implemented in any method or technology for storage of information, such as computer readable instructions, data structures, program modules, or other data. Examples of computer-readable storage media include RAM, ROM, EEPROM, flash memory, or other memory technology, CD-ROM, digital versatile disks (“DVD”) or other optical storage, magnetic cassettes, magnetic tape, magnetic disk storage or other magnetic storage devices, or any other medium which can be used to store the desired information and which can be accessed by a computer.

Communication methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

The display 406 can be any suitable display device, such as a monitor, screen, display, or the like, and can include a printer. The input device 408 can be, for example, a keyboard, mouse, touch screen, track ball, joystick, voice recognition system, or any combination thereof, or the like and can be used by the user to interact with a user interface.

The electrical stimulation system 412 can include, for example, a control module 414 (for example, an implantable pulse generator or external trial stimulator) and a lead 416 (for example, the leads illustrated in FIG. 1 or 2A-2I.) The electrical stimulation system 412 may communicate with the computer 400 through a wired or wireless connection or, alternatively or additionally, a user can provide information between the electrical stimulation system 412 and the computer 400 using a computer-readable medium or by some other mechanism. In some embodiments, the computer 400 may include part of the electrical stimulation system.

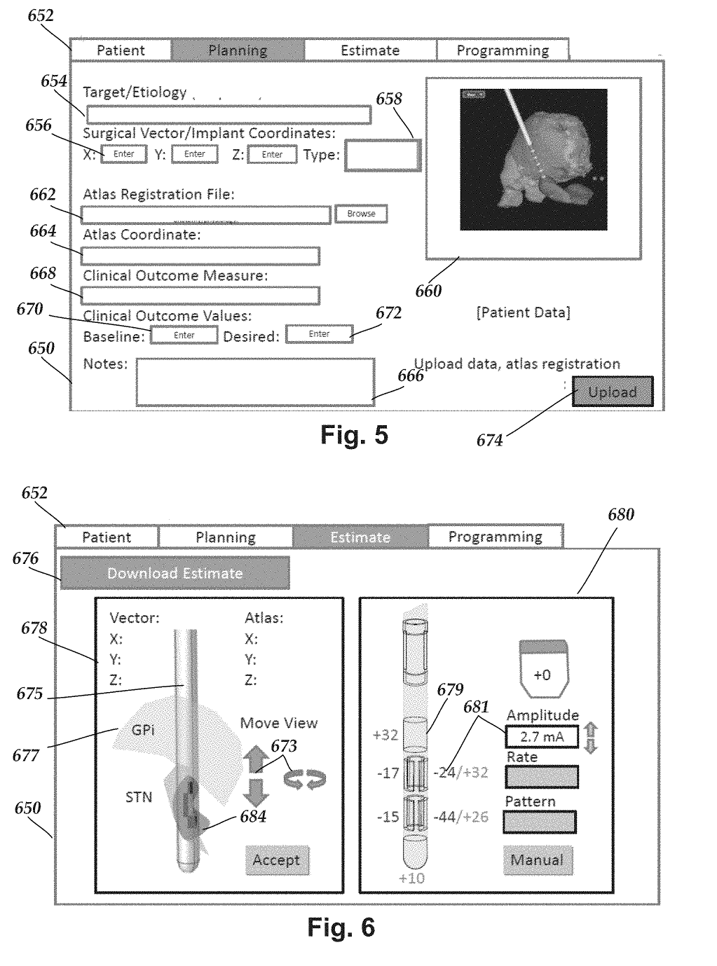

FIG. 5 illustrates a portion of a user interface 650 that can be used by a neurologist and neurosurgeon (or others) to plan implantation, report the results of implantation, and program an implanted neurostimulation lead and system. The user interface can be presented, for example, on the display 406 by the computer 400 and input can be received from the input device 408.

In at least some embodiments, the user interface 650 includes one or more modes that can be individually accessed. In the illustrated embodiment, the user interface 650 includes tabs 652 for the modes. In the illustrated embodiment, there are four tabs labeled “Patient”, “Planning”, “Estimate”, and “Programming”. Other mechanisms or arrangements for changing between modes can be used.

In the “Patient” mode (not shown), patient information can be entered or reviewed or, in some embodiments, a patient can be searched. Such patient information can include, but is not limited to, name, gender, age, ethnicity, address, primary care physician (or other physician), insurance information, medical record identifier, or the like.

FIG. 5 illustrates a screen of the user interface 650 for the “Planning” mode. The user interface 650 includes a target/etiology region 654 where the stimulation target, diagnosis, or etiology can be entered. In some embodiments, this entry can be made using pull-down menu or other selection arrangement. In other embodiments, the entry may be typed or otherwise entered into the region 654. Examples of the entries can be diagnoses, such as Parkinson's disease, depression, or the like; symptoms, such as tremor, rigidity, or the like; desired clinical responses, such as decreased tremor or rigidity or the like; or sites in the brain, such as substantia nigra, subthalamic nucleus, globus pallidus, or the like.

The user interface includes regions 656 for the surgical vector or implant coordinates. These entries may be planned entries provided by a neurologist, neurological planner, or neurosurgeon or they may be actual or estimated coordinates/vectors entered by the neurosurgeon after surgery. In some embodiments, the initial entries may be the planned entries that are then replaced with actual or estimated coordinates/vectors after implantation. The type region 658 may be used to specify what type of coordinates/vectors are provided, such as, for example, the center of the stimulation portion of the lead, the center of a stimulation field map that may be generated using the lead, a position of a particular electrode, or the like. Other features may include indication or selection of image frame type; usage or indication of frame coordinates; or image manipulation controls (for example, image registration, image stretching or rotation or other alterations, image filtering, highlighting of portions of the image, image segmentation into anatomical or other features, or the like). In some embodiments, the user interface may provide entries to specify a lead insertion point (e.g., a point on the cranium for insertion of the lead) or a target point for the distal end of the lead or the like.

In at least some embodiments, the system can request or receive information for surgical planning from separate surgical planning software or from a separate database, surgical planning station, or other source. Similarly, the system can request or receive information for diagnosis, etiology, symptom information, or the like from separate software or from a separate database or other source. In some embodiments, the system may automatically request such information and may be configurable with sources of the information. In some embodiments, the user interface 650 includes controls which the user can operate to obtain such information including, for example, a request control or source identification control or the like.

In some embodiments, the user interface 650 can also include an image region 660 that includes an actual image (e.g., a MRI or CT image) of a portion of the brain with a portion of the lead (either the actual lead or a model of the lead) or a simulated (or model) image of a portion of the brain with a model of the portion of the lead. This can provide a visual indication of the planned or actual site of implantation. In some embodiments, the image region 60 can permit a user to place a lead on the image or to rotate or move the image.

Other optional regions in the user interface 650 include an atlas registration file region 662 where reference to a file for a patient-specific or general atlas of the brain or region of the brain of interest can be inserted; an atlas coordinate region 664 where the type of coordinates (e.g., Talairach, MNI, or the like) used in the atlas can be indicated; and a note section 666 where notes from the neurologist or neurosurgeon can be entered. In addition, a clinical baseline or clinical outcome(s) section can include a region 668 to indicate a type of clinical outcome measure (for example, a UPDRS score, an accelerometer measurement or value for tremors or the like, a depression score, or any other suitable type of measurement); a baseline clinical outcome value 670 (for example, a measure prior to implantation) and a desire clinical outcome value 672 or a current clinical outcome value (not shown). There may also be an upload control 674 to initiate uploading this information to a server or the cloud or the Internet or another device. A download control (not shown) may also be provided to retrieve information for this (and, optionally, other) modes of the user interface 650.

FIG. 6 illustrates an “Estimate” mode of the user interface 650. In the “Estimate” mode, the system takes the information provided in the “Planning” mode and may use that information to produce an estimated position of the lead relative to anatomical or other features and may provide an estimated region of stimulation using one or more examples of stimulation parameters. In the illustrated user interface 650, an estimate control 676 is provided which, when activated, produces or obtains the estimated information. The estimated information may be generated by the system or may be obtained from another source, such as a server, programmer, or other system or software capable of producing the information.

In the illustrated user interface, 650, a first region 678 includes a model 675 of a portion of a lead and a model 677 of one or more regions or portions of the brain (or other anatomy) of the patient. In the illustrated embodiment, “GPi” refers to the patient's globus pallidus and “STN” refers to the patient's subthalamic nucleus. This estimate may be based on, for example, the surgical vector/implant coordinates and the atlas registration file indicated in the “Planning” mode of FIG. 5 which can be used to locate the lead relative to portions of the brain (or other anatomy) of the patient. In some embodiments, controls 673 can be provided to move or rotate the lead and other elements in the first region 678.

In the illustrated user interface, 650, a second region 680 includes a model 679 of a portion of a lead and electrodes with proposed stimulation parameters 681 which may include one or more of a stimulation amplitude, rate, or pattern, as well as a proposed relative distribution of the stimulation amplitude over the electrodes. The proposed stimulation parameters may be obtained as part of the estimate provided using the estimate control 676. In some embodiments, the proposed stimulation parameters may be stimulation parameters selected to provide stimulation to the target entered provided in the “Planning” mode. In other embodiments, the stimulation parameters may be trial stimulation parameters used by the neurosurgeon or may be stimulation parameters selected by the user. In the illustrated embodiment, the three values displayed to the left and right of the set of segmented electrodes correspond to the relative amplitude applied to the three electrodes. In the illustrated embodiment, a user can change the stimulation amplitude, but the rate and pattern are not shown or accessible. In some embodiments, the user may be allowed to access these parameters by actuation or by changing settings within the interface.

In some embodiments, an estimated region of stimulation (such as a stimulation field model) 684 can be included in either the first region 678 (as illustrated in FIG. 6) or second region 680. The region of stimulation 684 gives the user an estimate of the tissue that will be stimulated for the given set of stimulation parameters (e.g., selection of electrodes, stimulation amplitude, and distribution of the stimulation amplitude over the selected electrodes.) Examples of methods for determining the stimulation field model (or volume of activation) can be found in, for example, U.S. Pat. Nos. 7,346,282; 8,180,601; 8,209,027; 8,326,433; 8,589,316; 8,594,800; 8,606,360; 8,675,945; 8,831,731; 8,849,632; 8.958,615; 9.020,789; and U.S. Patent Application Publications Nos. 2009/0287272; 2009/0287273; 2012/0314924; 2013/0116744; 2014/0122379; 2015/0066111; and 2016/0030749, all of which are incorporated herein by reference.

The type of information that may be obtained using the estimate control 676 can depend on the information provided during planning or later. For example, when an etiology or target is provided during planning, the estimate can include the proposed stimulation parameters and visualization of the estimated region of stimulation. When one or more pre- or post-procedure images are included (optionally, with an atlas that may be registered to the one or more images), the visualization may also include anatomical structures and the spatial relationship between these structures and the lead or estimated region of stimulation.

When expected or observed therapeutic effects or side effects for particular stimulation parameters are included with the target/etiology, the system may determine a suggested set of stimulation parameters to start the programming session (and may also provide additional suggested sets based on the results obtained during the programming session.) Adding the one or more pre- or post-procedure images and atlas registration to the therapeutic effect/side effect information and target/etiology may permit the system to provide a real time stimulation estimate for a range of stimulation parameters. Examples of determination of such a stimulation estimate can be found in, for example, U.S. Provisional Patent Application Ser. No. 62/532,869 (Attorney Docket No. BSNC-1-650.0, filed Jul. 14, 2017, entitled “Systems and Methods for Estimating Clinical Effects of Electrical Stimulation”), incorporated herein by reference.

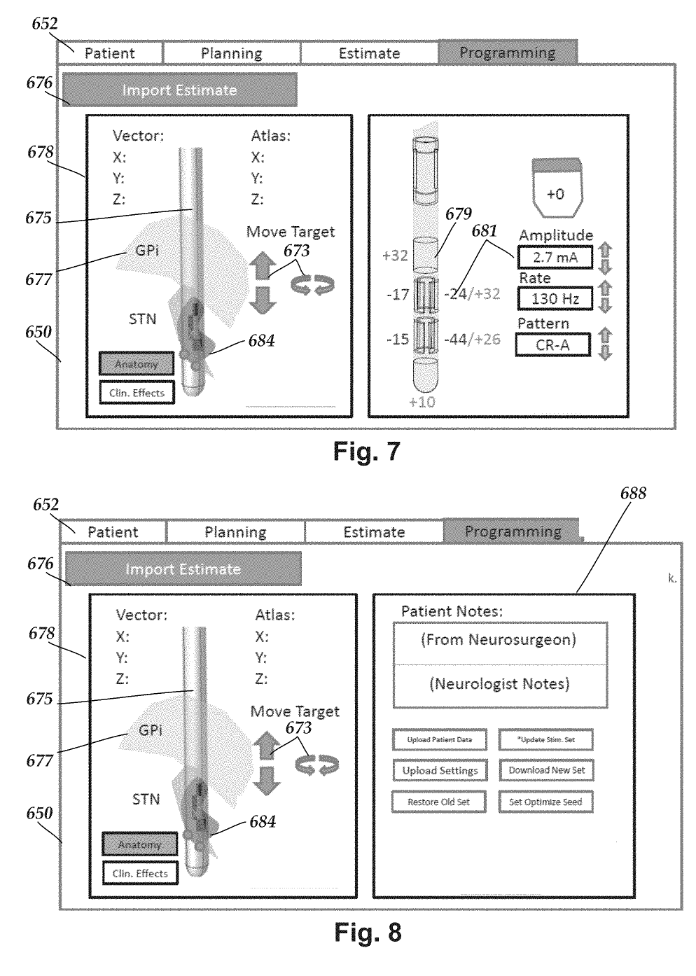

FIG. 7 illustrates a “Programming” mode of the user interface 650. In the “Programming” mode, a neurologist or programmer can test stimulation parameters to find suitable set of stimulation parameters to provide therapy using the implanted stimulation lead. The illustrated embodiment is similar to the “Estimate” mode except that more of the stimulation parameters (for example, the rate and pattern parameters) are available for adjustment or definition.

FIG. 8 illustrates another embodiment of a “Programming” mode of the user interface 650 which includes a notes section 688. For example, the neurosurgeon or neurologist may provide notes that are then accessible by operation of controls in this section. The notes section 688 may also include controls for uploading (or accessing) patient data, updating a set of stimulation parameters, update settings, download a set of stimulation parameters, restore an old set of stimulation parameters, or the like.

In some embodiments, the “Estimate” and “Programming” modes may be combined or may use the same interface screen.

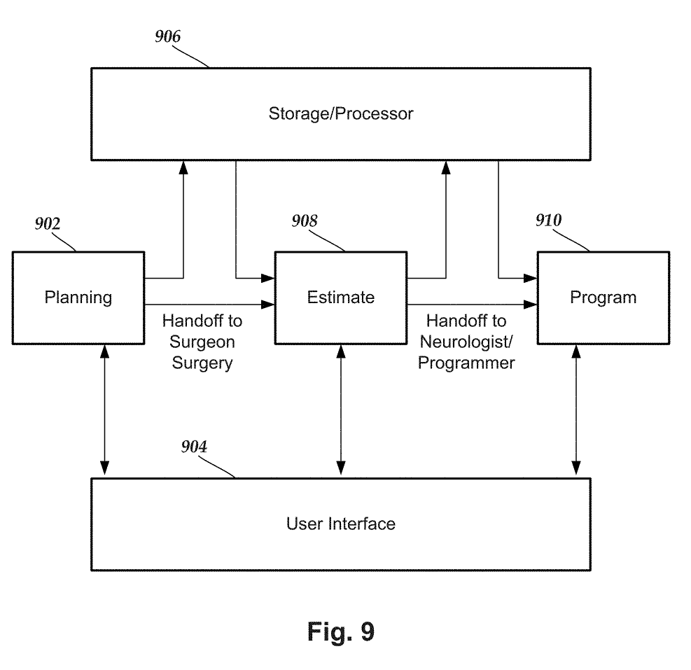

FIG. 9 illustrates a system and flow diagram illustrating data handling and hand off. As described above, the implantation of the stimulation lead is planned 902 by the neurologist or neurological planner using the user interface 904. The information generated during the planning is maintained at a storage/processor 906 which may also process the information to provide estimates for the therapy based on the planning. This storage/processor 906 may be provided on the same device that presents the user interface (such as a clinician programmer, remote control, mobile phone, tablet, computer, or the like) or may be provided on a different device or remotely (for example, in a remote or cloud server).

After planning 902, the process is handed off to the neurosurgeon who performs the surgery and enters information into the user interface 904 (such as the actual or estimated lead implantation site or results of a test stimulation or the like) which provided to storage/processor 906. The information from the planning 902 and the surgeon can be used to generate an estimate 908 which can be presented on the user interface 1104 and provided to the storage/processor. This can be handed off to the neurologist or programmer to program 910 the electrical stimulation system using the user interface 904 and the information from the storage/processor 906.

The above specification provides a description of the invention and its manufacture and use. Since many embodiments of the invention can be made without departing from the spirit and scope of the invention, the invention also resides in the claims hereinafter appended.

What is claimed as new and desired to be protected by Letters Patent of the United States is:

1. A system for planning implantation of an electrical stimulation lead of an electrical stimulation system and programming the electrical stimulation system after implantation, the system comprising:
a display; and
a processor that executes instructions to perform actions, comprising:
receiving an anatomical target for stimulation;
receiving at least one pre-implantation image including the anatomical target;
receiving or determining a target implantation site based on the anatomical target;
providing an indication of the target implantation site on the display to aid implantation of the electrical stimulation lead;
after implantation of the electrical stimulation lead, receiving or determining an estimate of the actual implantation site; and
providing an initial set of stimulation parameters for programming the electrical stimulation system based on the estimate of the actual implantation site and the anatomical target.

2. The system of claim 1, wherein the instructions further comprise receiving results of trial stimulation conducted following the implantation, wherein providing an initial set of stimulation parameters comprises providing the initial set of stimulation parameters for programming the electrical stimulation site based on the estimate of the actual implantation site, the anatomical target, and the results of the trial stimulation.

3. The system of claim 2, wherein the results of the trial stimulation comprise at least one set of trial stimulation parameters and, for at least one of the at least one set of trial stimulation parameters, an indication of a clinical result obtained when using that set of trial stimulation parameters.

4. The system of claim 1, wherein the instructions further comprising receiving or determining a registration of the at least one pre-implantation image to an anatomical atlas.

5. The system of claim 1, wherein receiving an anatomical target for stimulation comprises receiving an etiology and determining the anatomical target for stimulation based on the etiology.

6. The system of claim 1, wherein receiving an anatomical target for stimulation comprises receiving a target stimulation effect and determining the anatomical target for stimulation based on the target stimulation effect.

7. The system of claim 1, wherein the instructions further comprising receiving at least one post-implantation image of the anatomical site and the implanted electrical stimulation lead.

8. A system for planning implantation of an electrical stimulation lead of an electrical stimulation system and programming the electrical stimulation system after implantation, the system comprising:
a display; and
a processor that executes instructions to generate a user interface, the user interface comprising:
one or more input controls for inputting patient information
a target region for inputting or selecting an anatomical target or etiology for stimulation;
an image region for displaying at least one pre-implantation image including the anatomical target or anatomical region associated with the etiology;
an implantation site region for inputting or selecting an actual, estimated, or planned implantation site for a stimulation lead;
an estimate region for displaying an estimate of a position of the stimulation lead at the actual, estimated or planned implantation site relative to one or more anatomical features; and
a programming region for inputting or modifying stimulation parameters for programming the electrical stimulation system for delivery of electrical stimulation using the electrical stimulation lead.

9. The system of claim 8, wherein the user interface further comprises a note region for inputting notes by the user regarding implantation or programming.

10. The system of claim 8, wherein the estimate region further comprises displaying, relative to the stimulation lead, an estimated region of stimulation based on a set of stimulation parameters.

11. The system of claim 10, the processor is configured to alter the estimated region of stimulation when the stimulation parameters are modified.

12. The system of claim 8, wherein the user interface further comprises an atlas registration region for accessing an anatomical atlas.

13. The system of claim 12, the processor is configured to register the anatomical atlas to the at least one pre-implantation image.

14. The system of claim 8, wherein the image region is further configured for displaying at least one post-implantation image.

15. A system for planning implantation of an electrical stimulation lead of an electrical stimulation system and programming the electrical stimulation system after implantation, the system comprising:
a display; and
a processor that executes instructions to perform actions, comprising:
receiving an anatomical target or etiology for stimulation;
receiving at least one pre-implantation image;
after implantation of the electrical stimulation lead, receiving an estimate of the implantation site;
presenting a display of a model of the electrical stimulation lead and anatomical features based on the estimate of the implantation site; and
providing an initial set of stimulation parameters for programming the electrical stimulation system based on the estimate of the implantation site.

16. The system of claim 15, wherein the instructions further comprise receiving results of trial stimulation conducted following the implantation, wherein providing an initial set of stimulation parameters comprises providing the initial set of stimulation parameters for programming the electrical stimulation site based on the estimate of the actual implantation site, the anatomical target, and the results of the trial stimulation.

17. The system of claim 16, wherein the results of the trial stimulation comprise at least one set of trial stimulation parameters and, for at least one of the at least one set of trial stimulation parameters, an indication of a clinical result obtained when using that set of trial stimulation parameters.

18. The system of claim 15, wherein the instructions further comprising receiving or determining a registration of the at least one pre-implantation image to an anatomical atlas.

19. The system of claim 15, wherein receiving an anatomical target or etiology for stimulation comprises receiving the etiology and determining the anatomical target for stimulation based on the etiology.

20. The system of claim 15, wherein the instructions further comprising receiving at least one post-implantation image of the anatomical site and the implanted electrical stimulation lead.
